(19) United States
(12) Patent Application Publication
(10) Pub. No.: US 2008/0161563 A1
(43) Pub. Date: Jul. 3, 2008

(54) Reduction methodologies for converting ketal acids, salts, and esters to ketal alcohols

(76) Inventors: Robert J. Topping, Longmont, CO (US); Charles E. Tucker, Superior, CO (US); Gregory P. Withers, Boulder, CO (US)

Correspondence Address: ROCHE PALO ALTO LLC;Patent Law Department, M/S A2-250, 3431 Hillview Avenue, Palo Alto, CA, 94304, US

(21) Appl. No.: 12/004,191

(22) Filed: Dec. 20, 2007

Related U.S. Application Data
(60) Provisional application No. 60/877,878, filed on Dec. 29, 2006.

(51) Int. Cl. C07D 241/20; C07D 319/08

(52) U.S. Cl. 544/336; 549/333

(57) ABSTRACT

The present invention relates to methods of reducing ketal acids, salts and esters to form corresponding ketal alcohols. More particularly, the reducing methods convert the ketal acids, salts, or esters to ketal alcohols by using a reducing agent that comprises a hydride that comprises one or more alkoxy moieties. The ketal alcohol is prepared in a hydrophobic reagent. This is purified by washing the hydrophobic reagent with one or more water washes. Because the ketal alcohol has some water solubility, the water washes are back-extracted with a hydrophobic solvent to recover additional ketal alcohol from such one or more water washes. The alcohol products are useful in many applications such as intermediates in the synthesis of pharmacologically important molecules.

### PRIORITY CLAIMS

The present non-provisional patent Application claims benefit from U.S. Provisional Patent Application having Ser. No. 60/877,878, filed on Dec. 29, 2006, by Topping et al., and titled REDUCTION METHODOLOGIES FOR CONVERTING KETAL ACIDS, SALTS, AND ESTERS TO KETAL ALCOHOLS, wherein the entirety of said provisional patent application is incorporated herein by reference.

### FIELD OF THE INVENTION

The present invention relates to methods of reducing ketal acids, salts and esters to form corresponding ketal alcohols. More particularly, the reducing methods convert the ketal acids, salts, or esters to ketal alcohols by using a reducing agent that comprises a hydride that comprises one or more alkoxy moieties. The alcohol products are useful in many applications such as intermediates in the synthesis of pharmacologically important molecules.

### BACKGROUND OF THE INVENTION

Glucokinase (GK) is one of four hexokinases that are found in mammals [Colowick, S. P., in The Enzymes, Vol. 9 (P. Boyer, ed.) Academic Press, New York, N.Y., pages 1-48, 1973]. The hexokinases catalyze the first step in the metabolism of glucose, i.e., the conversion of glucose to glucose-6-phosphate. Glucokinase has a limited cellular distribution, being found principally in pancreatic β-cells and liver parenchymal cells. In addition, GK is a rate-controlling enzyme for glucose metabolism in these two cell types that are known to play critical roles in whole-body glucose homeostasis [Chipkin, S. R., Kelly, K. L., and Ruderman, N. B. in Joslin's Diabetes (C. R. Khan and G. C. Wier, eds.), Lea and Febiger, Philadelphia, Pa., pages 97-115, 1994]. The concentration of glucose at which GK demonstrates half-maximal activity is approximately 8 mM. The other three hexokinases are saturated with glucose at much lower concentrations (<1 mM).

Therefore, the flux of glucose through the GK pathway rises as the concentration of glucose in the blood increases from fasting (5 mM) to postprandial (≈10-15 mM) levels following a carbohydrate-containing meal [Printz, R. G., Magnuson, M. A., and Granner, D. K. in Ann. Rev. Nutrition Vol. 13 (R. E. Olson, D. M. Bier, and D. B. McCormick, eds.), Annual Review, Inc., Palo Alto, Calif., pages 463-496, 1993]. These findings. contributed over a decade ago to the hypothesis that GK functions as a glucose sensor in β-cells and hepatocytes (Meglasson, M. D. and Matschinsky, F. M. Amer. J. Physiol. 246, E1-E13, 1984).

In recent years, studies in transgenic animals have confirmed that GK does indeed play a critical role in whole-body glucose homeostasis. Animals that do not express GK die within days of birth with severe diabetes while animals overexpressing GK have improved glucose tolerance (Grupe, A., Hultgren, B., Ryan, A. et al., Cell 83, 69-78, 1995; Ferrie, T., Riu, E., Bosch, F. et al., FASEB J., 10, 1213-1218, 1996). An increase in glucose exposure is coupled through GK in β-cells to increased insulin secretion and in hepatocytes to increased glycogen deposition and perhaps decreased glucose production.

The finding that type II maturity-onset diabetes of the young (MODY-2) is caused by loss of function mutations in the GK gene suggests that GK also functions as a glucose sensor in humans (Liang, Y., Kesavan, P., Wang, L. et al., Biochem. J. 309, 167-173, 1995). Additional evidence supporting an important role for GK in the regulation of glucose metabolism in humans was provided by the identification of patients that express a mutant form of GK with increased enzymatic activity. These patients exhibit a fasting hypoglycemia associated with an inappropriately elevated level of plasma insulin (Glaser, B., Kesavan, P., Heyman, M. et al., New England J. Med. 338, 226-230, 1998). While mutations of the GK gene are not found in the majority of patients with type II diabetes, compounds that activate GK, and thereby increase the sensitivity of the GK sensor system, would still be useful in the treatment of the hyperglycemia characteristic of all type II diabetes. Glucokinase activators would increase the flux of glucose metabolism in β-cells and hepatocytes, which would be coupled to increased insulin secretion. Such agents would be useful for treating type II diabetes.

The following glucokinase activator (referred to herein as the compound of Formula I)

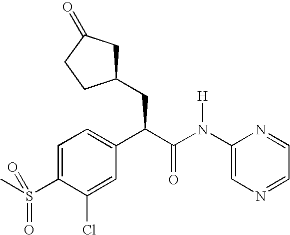

and its isopropanol (IPA) solvate of the formula:

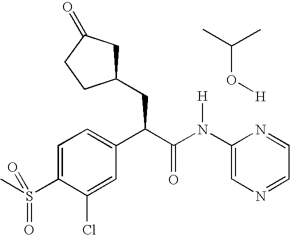

are under evaluation as a potentially new therapy for the treatment of Type 2 diabetes.

The compound of Formula I has also been described in PCT Patent Publication No. WO 03/095438 as well as in the co-pending U.S. Nonprovisional application Ser. No. 11/583,971, corresponding to U.S. Publication No. 2007/0129554, titled ALPHA FUNCTIONALIZATION OF CYCLIC, KETALIZED KETONES AND PRODUCTS THEREFROM, bearing Attorney Docket No. RCC0021/US, and filed Oct. 19, 2006, in the names of Harrington et al (hereinafter Application A); U.S. Provisional Application No. 60/791,256, titled PROCESS FOR THE PREPARATION OF A GLUCOKINASE ACTIVATOR, bearing Attorney Docket No. 23026, and filed Apr. 12, 2006 in the names of Andrzej Robert Daniewski et al., (hereinafter Application B); and U.S. Provisional Patent Application No. 60/877,877, titled EPIMERIZATION METHODOLOGIES FOR RECOVERING STEREOISOMERS IN HIGH YIELD AND PURITY, bearing Attorney Docket No. RCC0031/P1, and filed Dec. 29, 2006 in the name of Robert J. Topping (hereinafter Application C). All of these patent documents are incorporated herein by reference in their respective entireties for all purposes.

Application B schematically shows and describes a multi-step reaction scheme in which the compound of Formula I is manufactured from a ketal acid starting material in nine main reaction steps. In step 1 of this reaction scheme, a ketal acid is reduced to form a ketal alcohol. The reduction involved using either lithium aluminum hydride or Sodium Dihydro-bis-(2-Methoxyethoxy) Aluminate (available under trade designations RED-AL or VITRIDE). In the former cases, filtration was used to isolate the alcohol product from the salt by-products. Unfortunately, such an approach is not suitable for large scale production inasmuch as it is difficult on a larger scale to effectively isolate the alcohol from the salts using filtration techniques. Additionally, a yield of only 85% was achieved when using the RED-AL (also known as VITRIDE) reducing agent, which is lower than would be desired.

### SUMMARY OF THE INVENTION

The present invention relates to methods of reducing ketal acids, salts and esters to form corresponding ketal alcohols. More particularly, the reducing methods convert the ketal acids, salts, or esters to ketal alcohols by using a reducing agent that comprises a hydride that comprises one or more alkoxy moieties. This is followed up by extracting the ketal alcohol into a suitable organic phase, e.g., toluene, from extraction mixtures comprising an organic phase and an aqueous phase. The aqueous phase(s) are back-washed one or more times with the organic solvent in order to recover additional ketal alcohol product from the aqueous phase(s), significantly upgrading the yield of the ketal alcohol. Combining the use of such reducing agents with the back extractions can increase yields to over 90%, e.g., 95% in representative embodiments, as compared to yields of only 85% when such extractions are not used. The resultant alcohol products are useful in many applications such as intermediates in the synthesis of pharmacologically important molecules.

Hydride reducing agents that comprise one or more alkoxy moieties, especially those further including metal oxide constituents are very soluble in hydrophobic solvents such as toluene and the like. This allows the reducing methodologies to be carried out in a hydrophobic environment so that subsequent isolation of the ketal alcohol from salt by-products is easily achieved via one or more extractions between organic and aqueous phases. The ketal alcohol tends to be extracted into the organic phase(s), while the salt by-products are highly soluble in the aqueous phase. However, the by-products of the reduction tend to make the ketal alcohol more soluble in the aqueous phase. Back washing the aqueous phase(s) one or more times with an organic solvent helps to recover some of the solubilized ketal alcohol from the aqueous phase that would otherwise be lost. The ketal alcohol can be obtained in high yield and purity in this way without having to try to separate the alcohol from the salts via filtration. Consequently, the methods of the present invention are very suitable for large scale production.

In one aspect, the present invention relates to a method of making a ketal alcohol. A ketal acid is provided. The ketal acid is contacted with a reducing agent, said reducing agent comprising a hydride comprising one or more alkoxy moieties.

In one aspect, the present invention relates to a method of making a ketal alcohol. A ketal acid is provided. The ketal acid is contacted with a reducing agent in a hydrophobic solvent under conditions effective to convert the ketal acid to a ketal alcohol. The reducing agent comprises a hydride comprising one or more alkoxy moieties. The reduction reaction is quenched. The hydrophobic solvent containing the ketal alcohol is washed with one or more water washes, wherein at least a portion of the water washes include a portion of the ketal alcohol product. At least said portion of the one or more water washes is/are back-extracted with a hydrophobic solvent to extract ketal alcohol from said portion into said back-extracting hydrophobic solvent.

In another aspect, the present invention relates to a method of making a pharmacologically active compound A ketal acid is provided. The ketal acid is contacted with a reducing agent in a hydrophobic solvent under conditions effective to convert the ketal acid to a ketal alcohol. The reducing agent comprises a hydride comprising one or more alkoxy moieties. The reduction reaction is quenched. The hydrophobic solvent containing the ketal alcohol is washed with one or more water washes, wherein at least a portion of the water washes include a portion of the ketal alcohol product. At least said portion of the one or more water washes is/are back-extracted with a hydrophobic solvent to extract ketal alcohol from said portion into said back-extracting hydrophobic solvent. The ketal alcohol is used to make the pharmacologically active compound.

### BRIEF DESCRIPTION OF THE DRAWINGS

The above mentioned and other advantages of the present invention, and the manner of attaining them, will become more apparent and the invention itself will be better understood by reference to the following description of the embodiments of the invention taken in conjunction with the accompanying drawings, wherein:

FIG. 1 shows one embodiment of an aromatic cation that may be incorporated into a ketal salt.

FIG. 2a shows a preferred, chiral (S) aromatic cation that may be incorporated into a ketal salt.

FIG. 2b shows an alternative, chiral (R) aromatic cation that may be incorporated into a ketal salt.

FIG. 3 shows an illustrative reaction scheme in which a ketal acid, salt or ester is converted to a ketal alcohol.

FIG. 4 shows a more preferred reaction scheme in which a ketal acid, salt or ester is converted to a ketal alcohol.

FIG. 5 shows an example of a reaction scheme in which a particular ketal acid, salt or ester is converted to a corresponding ketal alcohol.

FIG. 6 shows an illustrative synthesis scheme for making the compound of Formula I and its IPA solvate in which principles of the present invention are used to convert a ketal acid to a ketal alcohol via reduction; and

FIG. 7 shows an alternative synthesis scheme for making the compound of Formula I and its IPA solvate in which principles of the present invention are used to convert a ketal acid to a ketal alcohol via reduction.

### DETAILED DESCRIPTION

The embodiments of the present invention described below are not intended to be exhaustive or to limit the invention to the precise forms disclosed in the following detailed description. Rather the embodiments are chosen and described so that others skilled in the art may appreciate and understand the principles and practices of the present invention.

In one aspect, the present invention involves converting a ketal functional, carboxylic acid, or salt or ester thereof, to the corresponding alcohol. Generally, this conversion involves reducing one or more carboxylate moieties of the material to form one or more corresponding hydroxyl moieties. The reaction is carried out in a suitable hydrophobic solvent such as toluene (organic phase). Ketal alcohol product recovered in the organic phase via extraction by washing the organic phase one or more times with water. Because the ketal alcohol has some degree of solubility in water, the aqueous wash(es) are back-extracted with one or more organic washes (e.g., toluene) to recover ketal alcohol from the aqueous wash(es).

The ketal functional, carboxylic acid (or salt or ester generally refers to a material comprising at least one ketal moiety and at least one carboxylic acid moiety, or salt or ester thereof. The carboxylic acid (or salt or ester) moiety generally has the formula —C(O)OM, wherein M is hydrogen, a monovalent organic substituent, or a suitable cation such as sodium, potassium, lithium, ammonium, an aromatic cation, combinations of these, and the like. The monovalent organic substituent may comprise an aliphatic and/or aromatic hydrocarbyl moiety that may be linear, cyclic, or branched. In some embodiments, the embodiment of M in the form of a hydrocarbyl moiety includes from 1 to 10 carbon atoms, and often may be methyl or ethyl. In some modes of practice, M is a nitrogen-containing cation such as a compound of the formula N—(R^{o})₄⁺, wherein each R^{o} independently is hydrogen and/or an achiral or chiral monovalent moiety that may be substituted or unsubstituted; or linear, branched, or cyclic. In some embodiments, two or more of the R^{o} moieties may be co-members of a ring structure.

Preferably, at least one R⁰ moiety includes an aromatic ring linked to the N by a divalent linking group such as an alkylene moiety of 1 to 15 carbon atoms. For example, a particularly preferred nitrogen-containing moiety suitable for M is an aromatic cation that has the formula

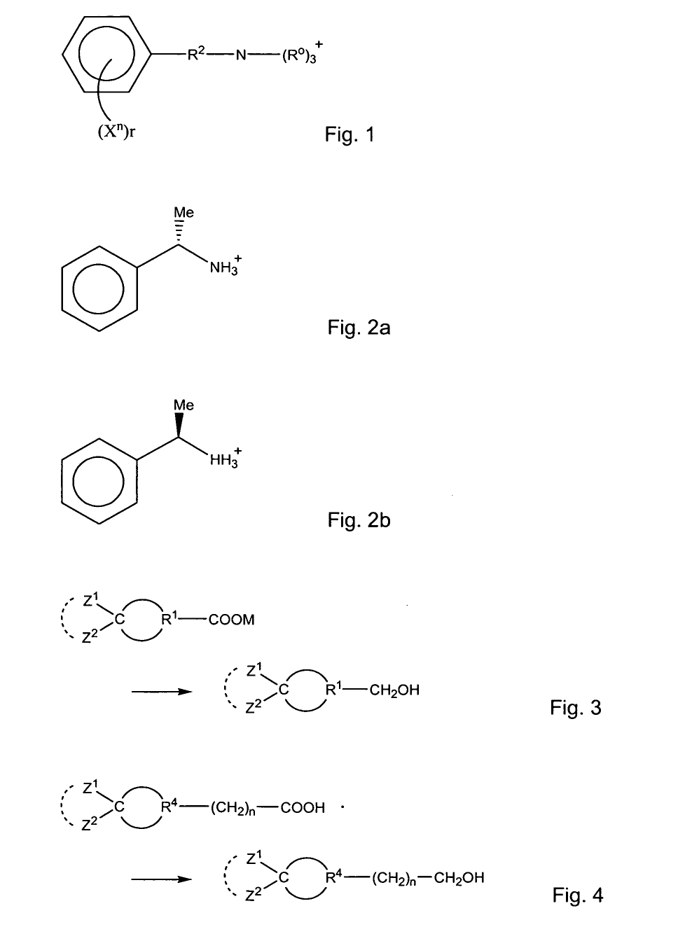

(Ar¹)ₚ—R²—N—(R⁰)_{q}⁺

wherein Ar¹ is a monovalent moiety comprising an aromatic ring; R² is a straight, branched, or cyclic alkylene moiety of 1 to 15, preferably 1 to 6 carbon atoms; p is 1 to 4, preferably 1; R⁰ is as defined above; q is 0 to 3; and p+q is 4. Preferably, the aromatic cation has the formula according to FIG. 1, wherein R^{o} and R² are as defined above; each Xⁿ is a monovalent substituent or co-member of a ring structure with another substituent; and r is 0 to 5, preferably 0. More preferably, the aromatic cation is an (R) and/or (S) chiral material having the formula according to FIG. 2a or FIG. 2b. In the context of a synthesis of the compound of Formula I, the aromatic cation desirably is the (S) form according to FIG. 2a.

A ketal moiety is a functional group that includes a carbon atom bonded to both —OZ¹ and —OZ² groups, wherein each of Z¹ and Z² independently may be a wide variety of monovalent moieties or co-members of a ring structure. In representative embodiments, Z¹ and Z² alone or as co-members of a ring structure are linear, branched, or cyclic alkyl(ene); preferably alkyl(ene) of 1 to 15, preferably 2 to 5 carbon atoms. The divalent, branched alkylene backbone associated with neopentyl glycol is a preferred structure when Z¹ and Z² are co-members of a ring structure.

A ketal is structurally equivalent to an acetal, and sometimes the terms are used interchangeably. In some uses, a difference between an acetal and a ketal derives from the reaction that created the group. Acetals traditionally derive from the reaction of an aldehyde and excess alcohol, whereas ketals traditionally derive from the reaction of a ketone with excess alcohol. For purposes of the present invention, though, the term ketal refers to a molecule having the resultant ketal/acetal structure regardless of the reaction used to form the group.

If the ketal material is first provided in the form of a salt or acid, it is first desirable to convert the salt or ester to an acid form. Direct reduction of the salt could tend to generate undesirable byproducts such as the corresponding free amine. This would contaminate the resultant ketal-alcohol during workup, and the amine could be very difficult to separate at that point. Converting the salt or ester to the acid may be accomplished using any conventional technique.

For instance, when the ketal is supplied in a salt form, an acid may be used for salt cleavage. One way to accomplish this is to disperse the salt in a suitable organic solvent that is immiscible with water. It is convenient if the solvent is the same as the organic solvent to be used for reduction. Toluene is one illustrative solvent that may be used for both the salt cleavage and the reduction. It is also convenient to use aqueous acid. The aqueous acid may be added to the salt containing mixture or the ketal can be slowly added to the aqueous acid. In any case, mixing occurs with agitation. The resultant two-phase mixture is allowed to settle. The ketal acid product will tend to be more soluble in the organic phase, while salt by-products will tend to be in the aqueous phase. The two phases are easily separated to recover the ketal acid in the organic phase. Optionally, the organic phase can be washed one or more additional times with water and/or the aqueous phase can be washed one or more additional times with organic solvent, to further enhance the purity and yield of the ketal acid. At the end of any such washes, the organic phases containing the ketal acid can be combined, optionally concentrated, optionally isolated, and then taken forward to carry out the reduction reaction.

The acid used for salt or ester cleavage to yield the ketal acid should be of moderate strength. If the acid is too strong, the acid could degrade the ketal moiety. Examples of suitable acids of moderate strength that are reasonably compatible with the ketal group include organic acids such as citric acid, acetic acid, succinic acid, tartaric acid, malonic acid, malic acid, and combinations of these, and the like. Desirably, only enough acid is added to ensure that cleavage of as much of the salt or ester is achieved as is practical, inasmuch as too much excess acid risks degradation of the ketal group even when using an acid of moderate strength.

In the practice of the present invention, the reduction of the ketal functional carboxylic acid (or salt or ester thereof) occurs in a reaction medium comprising a reducing agent that is a hydride comprising at least one alkoxy moiety and at least one additional constituent. The reducing agent and the ketal alcohol may be combined all at once or more desirably gradually as the reduction reaction progresses. Preferably, the ketal-acid is added to the excess reducing agent.

In representative embodiments, each alkoxy moiety of the reducing agent generally independently has the formula —OR³—, wherein R³ is a divalent aliphatic and/or aromatic hydrocarbyl. Desirably, each R³ is a linear, branched or cyclic alkylene moiety containing 1 to 10 carbon atoms, often 1 to 6 carbon atoms.

The at least one other constituent included in the reducing agent comprises one or more atoms such as B, Li, Na, K, Mg, Ca, Al, selenium, bismuth, antimony, tellurium, silicon, lead, germanium, arsenic, nitrogen, tin, polonium, combinations of these, and the like. These atoms may be present in any suitable form, including as a constituent of an oxygen-containing species.

A particularly preferred kind of reducing agent is a hydride that comprises one or more alkoxy moieties and aluminum in a suitable form such as an aluminate. An example of one such reducing agent is sodium dihydro-bis-(2-methoxyethoxy)aluminate (also referred to as SDMA). SDMA is commercially available under the trade designation VITRIDE in solutions comprising about 69 weight percent of SDMA in toluene from Zeeland Chemicals). The VITRIDE reducing agent has a reductive strength that is somewhat in-between NaBH₄ and LiAlH₄. The VITRIDE material is a readily transferable liquid that is compatible with ketals, and is compatible with common, inexpensive, aprotic solvents such as toluene and the like.

The amount of reducing agent included in the reaction mixture may vary over a wide range. Generally, at least a modest excess of the reducing agent is included to help ensure that as much acid (or salt or ester thereof) is reduced as is practical to maximize yield. Using too much is wasteful of reagent and can make it more difficult to isolate the resultant product from the left over reducing agent. For instance, in the case of the VITRIDE reducing agent, each VITRIDE molecule has two available hydrides on a theoretical basis. Three hydrides are needed to reduce an acid to an alcohol. One hydride deprotonates the acid. A second hydride reduces the carboxylate to an aldehyde. A third hydride reduces the aldehyde to form the alcohol. Therefore, the molar ratio of the VITRIDE reducing agent to the acid is desirably at least 1.5:1 so that there are 3 equivalents of hydride for each equivalent of acid. Using such amounts achieves 95% yield of the ketal alcohol in representative embodiments. Using lesser amounts of the VITRIDE, e.g., 1.2 moles per mole of acid, achieves only 90% yields in other representative embodiments.

Using an excess of the reducing agent will tend to help achieve higher conversion of acid to alcohol. However, using too much excess reducing agent is not desirable, inasmuch as using more would require more quenching reagent. Also, using more would tend to produce more by-products to be removed. Hence, using too much reducing agent would make scale up less cost-efficient. Accordingly, in the case of VITRIDE, any excess of the VITRIDE should be slight, e.g., 1.55 moles of VITRIDE per mole of the acid. In one representative mode of practice according to a larger scale reaction, using 487.8 kg of a 70% solution of SDMA in toluene per 752.4 mol of a ketal acid was found to be suitable.

Because both the desired ketal acid starting material and the reducing agent are both soluble in a wide range of organic, aprotic, nonpolar solvents, the reducing conversion may occur in a wide range of solvents or solvent mixtures. Preferred solvents are aliphatic and/or aromatic hydrocarbon solvents inasmuch as such solvents are widely available and inexpensive. Toluene is a preferred organic solvent as it is widely available and cost effective. Toluene also facilitates aqueous workup after the conversion via conventional extraction techniques to separate the reduction by products from the desired alcohol product. The by-products tend to be more soluble in an aqueous phase, while the ketal alcohol product tends to be more soluble in the organic phase. THF could also be a good solvent, but workup will tend to be harder due to the greater water miscibility of THF.

The amount of solvent used to carry out the conversion of ketal acid to ketal alcohol can vary over a wide range. If there is too little solvent, though, then the intermediate ketal-acid can precipitate prior to the reduction, resulting in processing issues with the reduction. On the other hand, if there is too much solvent, then the reaction may take longer, the cost increases due to higher solvent usage and less vessel utilization. Balancing such concerns, the reaction medium to carry out the reduction generally may include from about 200 to about 1000 liters, more desirably 300 to 700 liters, of solvent per 50 to 500 kilograms of acid (or salt or ester).

The reduction reaction may be carried out at a wide range of temperatures over a wide range of time periods. For instance, the reaction may occur at any temperature ranging from just above 0° C. to 60° C. The reduction of carboxylic acids may be too sluggish (slow) below 0° C. to be practical, and degradation may tend to occur above 60° C. More desirably, the reaction mixture is maintained slightly chilled or near room temperature such as at a temperature in the range from about 5° C. to about 30° C., more commonly about 20° C. to about 30° C. Conducting the reduction at such moderately higher temperatures is preferred to enhance yield of the ketal alcohol without undue risk of degradation of the reactants or products.

The reaction desirably may occur for a time period in the range of from about a few minutes to several hours, desirably about 5 minutes to 8 hours.

After the reduction reaction is complete, it is desirable to quench the reduction reaction. According to one illustrative technique, this is accomplished by adding a base to the reaction mixture or vice versa. It is convenient to use aqueous base such as aqueous NaOH or the like. The ketal is best added slowly to the aqueous base because adding NaOH (aq) to the reaction could be more dangerous due to less control over hydrogen evolution. Additionally, adding aqueous NaOH to the ketal alcohol mixture tends to result in salt precipitation due to lack of water during the early phases of the quench. In any case, mixing occurs with agitation. The resultant two-phase mixture is allowed to settle. The ketal alcohol product will tend to be more soluble in the organic phase, while salt by-products will tend to be more soluble in the aqueous phase. The two phases are easily separated to recover the ketal alcohol in the organic phase. Optionally, the organic phase can be washed one or more additional times with water and/or the aqueous phase can be washed one or more additional times with organic solvent, to further enhance the purity and yield of the ketal alcohol. At the end of any such washes, the resultant organic phases can be combined, optionally concentrated, optionally isolated, and/or taken forward to carry further desired processing. For instance, the ketal alcohol may be reacted with suitable electrophiles to form iodides or tosylates useful for alkylation reactions in the course of synthesizing the compound of Formula I.

The conversion of ketal acid to ketal alcohol tends to yield by-products that can influence the effectiveness of extraction when aqueous work up is used to recover the ketal alcohol product in an organic phase, e.g., a toluene phase. For example, in the case of VITRIDE, quenching the reduction reaction tends to liberate 2-methoxyethanol. The presence of this alcohol by-product unfortunately enhances the water solubility of the ketal alcohol product to some degree. Although a major portion of the ketal alcohol product will be present in the organic phase upon aqueous workup, significant portions of the ketal alcohol nonetheless will be solubilized in the one or more aqueous washes used to remove salt by-products.

In short, in order to upgrade the yield of the ketal alcohol, it is desirable to minimize the loss of ketal alcohol into the aqueous washes during aqueous workup. To accomplish this, it is desirable to back-extract the aqueous phase(s) with one or more organic washes due to the slight water solubility of the ketal alcohol that is induced by the presence of reduction reaction by-products such as 2-methoxy ethanol.

Advantageously, therefore, the present invention uses one or more back extractions of the aqueous phase(s) to recover ketal alcohol from the aqueous phases that otherwise would be lost. Accordingly, after subjecting the alcohol product mixture to a first extraction among an organic phase and an aqueous phase, the aqueous phase can be subjected to one or more organic washes in order to recover additional ketal alcohol from the aqueous phase(s). The upgrade in yield is significant. In representative embodiments, yields of 95% are achieved when using such back extraction methods. In contrast, yields of only 85% are achieved without the back extractions.

Optionally, any of the organic layers obtained from the primary or back extractions can also be washed with water to further upgrade yields and/or purity, although such washes could cause some amounts of ketal alcohol to be lost to the aqueous layers unless such additional aqueous layers are also back extracted with an organic wash such as toluene.

One example of a reaction scheme by which a ketal functional carboxylic acid is reduced to form a corresponding ketal alcohol is provided by the reaction scheme shown in FIG. 3, wherein Z¹, Z², and M, are as defined above. R¹ is a trivalent moiety that links the carbon of the ketal group to the —COOM moiety of the acid (or salt or ester). R¹ may be aliphatic and/or aromatic, chiral or achiral, saturated or unsaturated, or substituted or unsubstituted. Preferably, R¹ is a saturated, chiral or achiral, aliphatic hydrocarbyl. comprising C and H atoms. More preferably, R¹ includes only carbon atoms and H substituents.

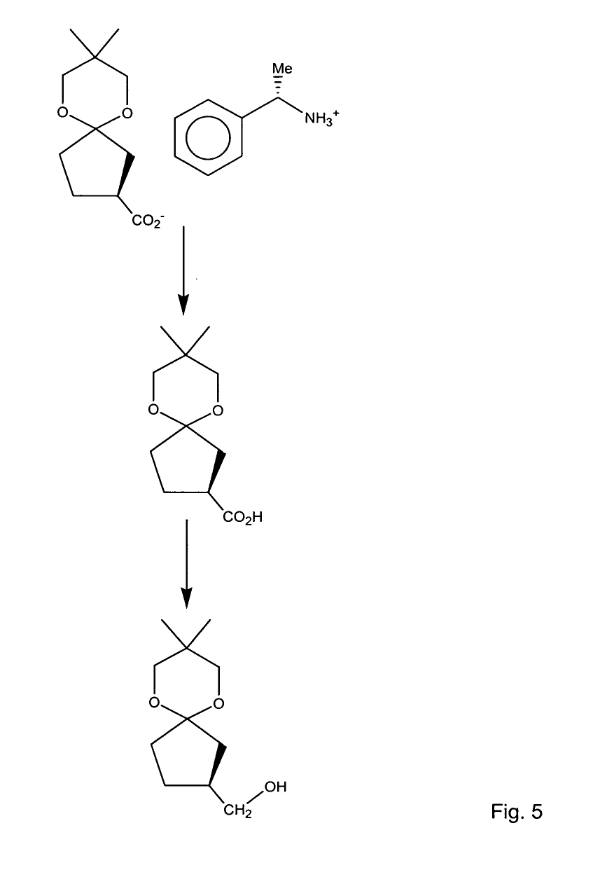

In particularly preferred embodiments, the reduction of the ketal acid to form a ketal alcohol may be represented by the reaction scheme in FIG. 4 wherein R⁴ together with the C atom of the ketal moiety form a cyclic moiety of 4 to 8, preferably 5 or 6 atoms; and n is 0 to 15, preferably 1 to 6. In preferred embodiments, R⁴ together with the C atom of the ketal moiety form a 5 or 6 membered ring in which all atoms of the ring structure are selected from C, O, S, and N, more preferably from C and O, and most preferably are C atoms. One specific example of a reduction reaction that first converts a ketal acid salt to the acid and then to a ketal alcohol is represented by the reaction scheme shown in FIG. 5.

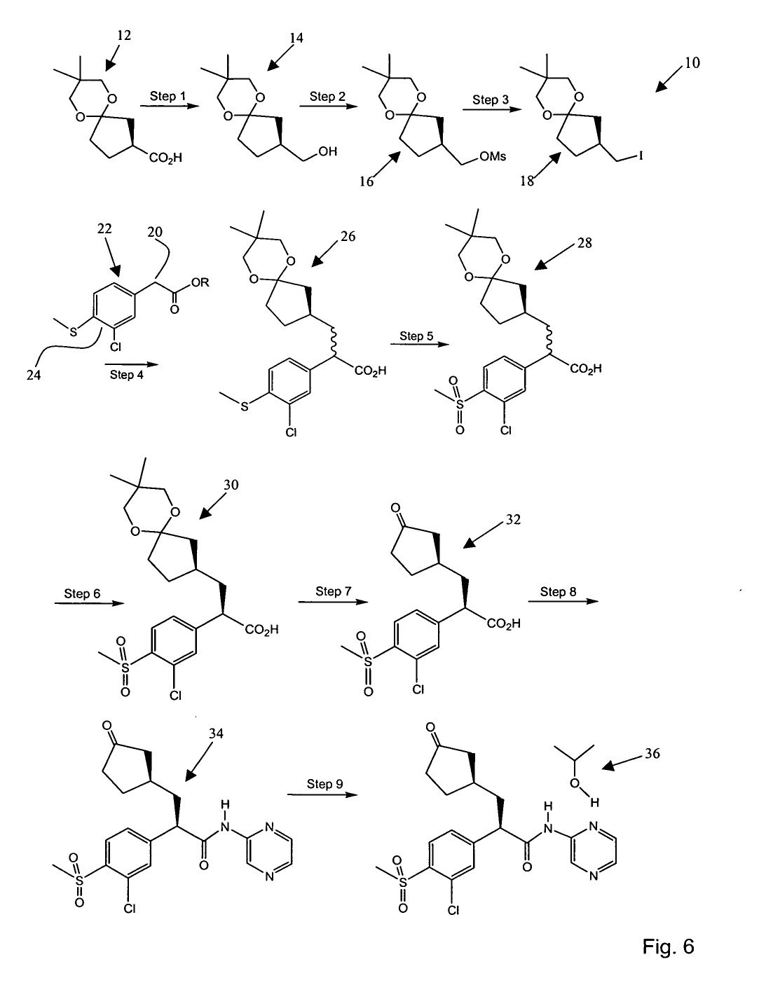

The principles of the present invention are beneficially used any time it is desired to convert a ketal acid (or salt or ester) to a ketal alcohol. As one example, the principles of the present invention may be used to synthesize pharmacologically active materials such as the compound of Formula I. FIG. 6 shows one such illustrative scheme 10. In step 1, a chiral (S) ketal acid 12 is reduced to the corresponding chiral (S) ketal alcohol 14. The principles of the present invention are used to carry out this reduction reaction. A specific example of this reaction is provided in the working examples below. The ketal acid 12 may be derived from an S-MBA salt precursor. An S-MBA ketal salt precursor (not shown) of ketal acid 12 may be prepared using the techniques described in co-pending Application A.

The remaining steps 2 through 9 may be carried out as described in co-pending Application B. As an overview of these steps as carried out in Application B, step 2 involves mesylating the ketal alcohol 14 to form the chiral ketal mesylate 16. The —OMs moiety of mesylate 16 has the formula

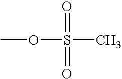

In step 3, the ketal mesylate 16 is converted to the chiral ketal iodide 18. In step 4, the iodide 18, which is a strong electrophile, is used to alkylate the alpha carbon 20 of the substituted, aromatic ester 22. The reaction is conveniently referred to as an alkylation inasmuch as the portion of iodide 18 that becomes directly linked to the ester 22 is the —CH₂— portion of the iodide 18. An aromatic substituent 24 that is pendant from the alpha carbon 20 of the ester 22 is believed to help stabilize an anion intermediate that results when a base in the alkylation reaction medium helps to de-protonate the alpha carbon 20. The R group of ester 22 is desirably ethyl.

The reaction product of step 4 is a mixture of (2R,3′R) and (2S,3′R) epimers 26. The thio moiety of these is oxidized in step 5 to form the corresponding sulfonylated epimers 28. The (2R,3′R) epimer 28 is carried forward in subsequent reaction steps, and so step 6 involves subjecting the epimers 28 to an epimerization reaction to convert the (2S,3′R) epimer to the desired (2R, 3′R) epimer 30. As an alternative option, this epimerization reaction may be carried out using the techniques as described in co-pending Application C inasmuch as the epimerization techniques of Application C may yield the desired epimer 30 in higher yield and purity.

Regardless of the epimerization technique used to carry out step 6, step 7 involves converting the ketal protecting group of epimer 30 to a ketone moiety to thereby form the sulfonylated, aromatic, ketone acid 32. In step 8, this acid 32 is reacted with a suitable co-reactant (not shown) to form the Formula I compound 34. In optional step 9, the compound of Formula I 34 is converted to its IPA solvate form 36.

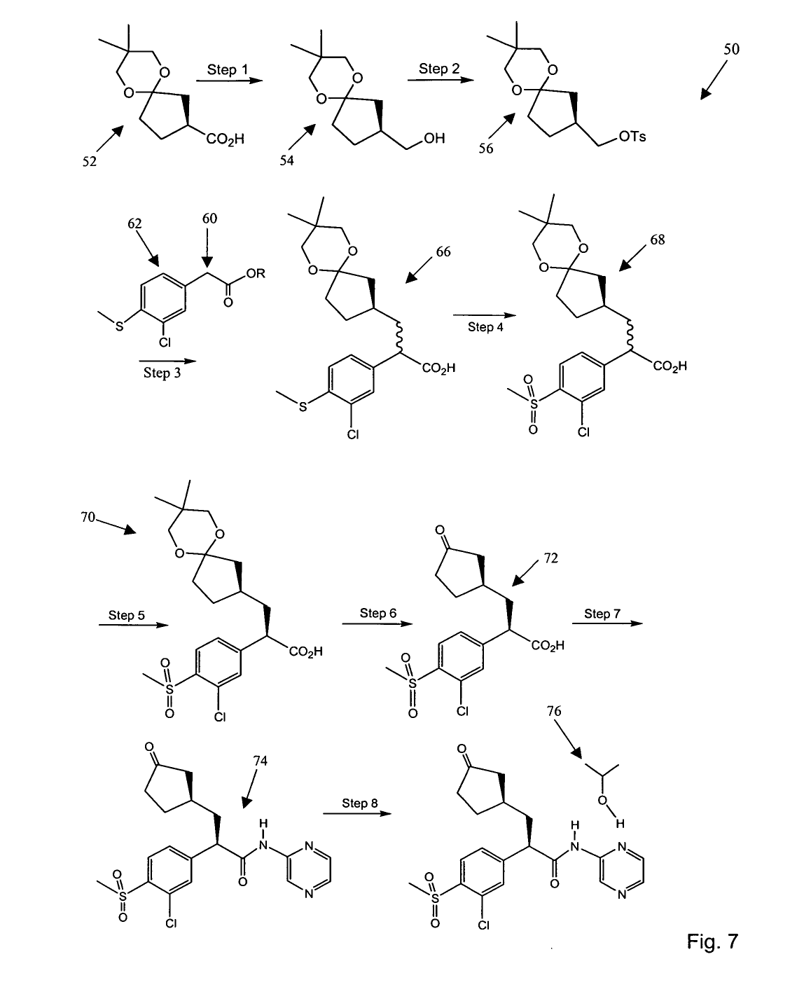

An alternative reaction scheme 50 that uses principles of the present invention to form the compound of Formula I is shown in FIG. 7. As an overview of the reaction scheme shown in FIG. 7, a chiral (S) ketal alcohol 52 is reduced to the corresponding chiral (S) ketal alcohol 54 in step 1. The principles of the present invention are used to carry out this reduction reaction. The ketal acid 52 may be derived from an S-MBA salt precursor. An S-MBA ketal salt precursor (not shown) of ketal acid 52 may be prepared using the techniques described in co-pending Application A. In step 2, the ketal alcohol 54 is converted to a tosylate 56. This may be accomplished using procedures as described in co-pending U.S. Provisional Patent Application No. 60/877,788, titled AROMATIC SULFONYLATED KETALS, bearing Attorney Docket No. RCC0032P1, filed Dec. 29, 2006 in the name of Robert J. Topping, the entirety of which is incorporated herein by reference for all purposes. The —OTs moiety has the formula

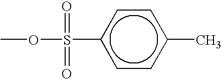

In step 3, the tosylate 56, a strong electrophile, is used to alkylate the alpha carbon 60 of the substituted, aromatic ester 62. This, too, may be accomplished as described in Application C. The R group of ester 62 is desirably ethyl. The remaining steps 4 through 8 in FIG. 7 may be carried out in the same manner as corresponding steps in FIG. 1 are carried out with respect to the mixture of (2R,3′R) and (2S, 3′R) epimers 66, sulfonylated epimers 68, the (2R, 3′R) epimer 70, the sulfonylated, aromatic, ketone acid 72, Formula I compound 74, the IPA solvate form 76.

All patents, published patent applications, other publications, and pending patent applications (including both provisional and nonprovisional applications) cited in this specification are incorporated by reference herein in their respective entireties for all purposes.

The present invention will now be described with reference to the following illustrative examples.

### EXAMPLE 1

### Applying Principles of Present Invention to Synthesis of Ketal Tosylate

### Salt Cleavage and (S)-Ketal-Acid Concentration

A 12,000 L glass-lined vessel was charged with 252.4 kg (752.4 mol) of an (S)-Ketal-acid, (S)-MBA salt precursor of ketal acid 12 of FIG. 6 or ketal acid 52 of FIG. 7 (this salt is described in Application A), followed by 1260 L (liters) toluene. The mixture (slurry) was cooled to 5° C. under nitrogen with agitation. To a 16,000 L glass-lined vessel was charged 212 L potable water followed by 318.0 kg 50% aqueous citric acid. The aqueous citric acid solution was cooled to 0° C. with agitation and then added to the ketal-acid salt slurry over 20 min while keeping the temperature of the reaction mixture. below 5° C. The two-phase reaction mixture was warmed to 13° C. and allowed to settle for 30 min. The lower aqueous layer was separated. To the aqueous citric acid layer was added 504 L toluene. The two-phase mixture was stirred for 15 min at 14° C. and allowed to settle for 49 min at 14° C. The lower aqueous layer was separated. The two toluene extracts containing the intermediate (S)-ketal acid were combined and 84 L potable water was added. The two-phase mixture was stirred for 17 min at 16° C. and the mixture allowed to settle for 60 min at 16° C. The lower aqueous layer was separated into a separate vessel.

To this aqueous solution was added 504 L toluene. The two-phase mixture was stirred for 20 min at 18° C. and allowed to settle for 30 min at 18° C. The lower aqueous layer was separated and combined with the aqueous citric acid solution and discarded. All of the toluene phases containing the (S)-ketal acid were combined and approximately 1,018 L of the toluene solution of the (S)-ketal-acid was transferred from the 12,000 L glass-lined vessel to a 2000 L Hastelloy vessel. Transfer of 1018 L of solution to the 2,000 L Hastelloy vessel provided for a significant amount of head space for the subsequent distillations to minimize the chance of bumping the batch into the vessel overheads.

The solution was concentrated via a feed-distillation under reduced pressure (30-40 mm pressure, vessel temperature ˜35° C. with a maximum bath temperature of 50° C.) until the volume of the (S)-ketal-acid solution reached 588 L. After the 12,000 L feed vessel is empty, the distillation was halted and the feed vessel rinsed with 84 L toluene to the distillation vessel. The distillation was then restarted and continued until the target volume was reached. The solution was sampled for Karl Fischer analysis and showed 0.007% contained water. The solution of the ketal-acid was then cooled to 10° C.

### (S)-Ketal-Acid Reduction

A 2,000 L glass-lined vessel was charged with 487.8 kg 70% Vitride solution in toluene followed by 441 L toluene with agitation. Approximately 9 L of toluene is used to flush out the charging dip leg after the Vitride charge. After the toluene charge, a recirculation loop containing a ReactIR™ monitoring instrument was started to monitor the reduction. The diluted Vitride solution was cooled to <5° C. The pre-cooled ketal-acid solution was transferred to the Vitride solution through a 20-micron polishing filter and ¼″ mass-flow meter at a rate of 2.0 kg/min. A mass flow meter was utilized as a safety precaution to minimize the risk of adding the ketal-acid at a rate that would generate hydrogen faster than could be safely handled in the reduction vessel. The reaction is very exothermic but the heat and hydrogen flow is completely controlled by the ketal-acid feed rate. A maximum addition rate was 2.2 kg/min. A polishing filter was used to prevent any residual salts from plugging the relatively small mass flow meter. A total of 581 kg of ketal-acid solution was transferred (density 0.959 kg/L)

The reaction temperature was maintained at <25° C. but with a target range of 20±5° C. throughout the ketal-acid addition. Running the reduction at a lower temperature (e.g. <10° C.) results in lower yields, presumably due to incomplete reduction. Maintaining ambient temperature for the reaction results in higher yields.

The vessel containing the ketal-acid solution was rinsed with 42 L toluene and the rinse transferred through the filter and mass-flow meter. The reduction reaction mixture was agitated for 70 min at 20-22° C. and sampled for reaction completion. The reaction was monitored by the ReactIR™ to check for the presence of the excess Vitride at the end of the reaction, but an HPLC sample was also taken to check for the presence of unreacted ketal-acid. To a 12,000 L glass-lined vessel was charged 596.8 kg 20% aqueous NaOH solution which was cooled to 2° C. with agitation. This quantity of 20% NaOH used for this batch (500 L, 600 kg) was determined by the minimum stirrable volume of the 12,000 L vessel used for the quench. The amount of NaOH can be reduced where practical concerns like this do not control. The recirculation loop used for the ReactIR™ was blown back into the reactor just prior to the quench.

The reaction mixture was then transferred to the aqueous NaOH solution through a ½″ mass flow meter while keeping the temperature of the quench mixture below 25° C. A maximum feed rate was set at 9 kg/min to control the hydrogen evolution. The addition time for this batch was 3 h with a maximum temperature of 16° C. (1,461 kg of reaction solution transferred).

The reduction reaction vessel was rinsed with 84 L toluene and the rinse transferred through the mass flow meter. The quench mixture was warmed to 16° C. and stirred for 1 h at 16-17° C. The agitation was stopped and the two-phase mixture allowed to settle for 1 h at 17° C. The lower aqueous layer containing the caustic aluminum salts was separated into another glass-lined vessel. To this aqueous solution was added 504 L toluene and the two-phase mixture stirred for 30 min at 21° C. and allowed to settle for 1 h at 21-22° C. The layers were separated and the two toluene layers containing the crude ketal-alcohol were combined followed by a 84 L toluene vessel rinse. To the aqueous layer was added 504 L toluene and the two-phase mixture stirred for 30 min at 18° C. and allowed to settle for 1 h at 18° C. The lower aqueous phase was separated and discarded (638 L for this batch).

The two toluene layers containing the crude ketal-alcohol were again combined followed by a 84 L toluene vessel rinse. To the total solution containing the intermediate ketal-alcohol was added 209 L potable water. The two-phase mixture was stirred for 38 min at 18-21° C. and allowed to settle for 1 h at 21° C. The water rinse serves to remove any residual salts, but also removes some of the 2-methoxyethanol liberated during the quench as well as some ketal-alcohol, thus requiring toluene back-extractions to minimize yield loss. The lower aqueous phase was separated and to it was added 211 L toluene. The two-phase mixture was stirred for 30 min at 24° C. and allowed to settle for 1 h at 24° C. The toluene layer was recombined with the bulk ketal-alcohol solution followed by a 84 L toluene vessel rinse. To the aqueous layer was added 210 L toluene. The two-phase mixture was stirred for 40 min at 23° C. and allowed to settle for 1.7 h at 23° C. The layers were separated and the aqueous layer discarded (399 L for this batch). The toluene layer was recombined with the bulk ketal-alcohol solution followed by a 84 L toluene vessel rinse. At this point, the ketal-alcohol solution was sampled for 2-methoxyethanol which was then monitored during the subsequent feed distillation. Approximately 1,018 L of the toluene solution of the ketal-alcohol was transferred from the 12,000 L glass-lined vessel to a 2000 L Hastelloy vessel. The solution was concentrated via a feed-distillation under reduced pressure (20 mm minimum pressure, vessel temperature ˜30-35° C. with a maximum bath temperature of 50° C.) until the volume of the ketal-alcohol reached 320 L. The ketal-alcohol solution was held for 1 h and any second-phase water present removed prior to starting the feed distillation.

After the initial feed distillation was complete, the ketal-alcohol solution was sampled for 2-methoxyethanol and water content. It was necessary to add additional toluene and continue the distillation to remove the 2-methoxyethanol to an acceptable level. A total of three additional toluene charges were required (100 L, 150 L and 500 L) with the final distillation volume being reduced to 220 L. The final 2-methoxyethanol content was 0.022% relative to the ketal-alcohol. Since the ketal-alcohol solution was to be eventually transferred back to the 12,000 L vessel for the tosylation reaction, no vessel rinse was performed during the distillation.

### Tosylation

The toluene solution of the ketal-alcohol was transferred to a 12,000 L glass-lined reactor followed by a 150 L toluene rinse. The 2,000 L Hastelloy reactor was vacuum dried and to it was charged 105.9 kg (944.0 mol) 1,4-diazabicyclo[2.2.2]octane (DABCO) followed by 605 L toluene. The mixture was stirred for 1.2 h at 15-16° C. until the solids were dissolved. The DABCO solution was combined with the solution of the ketal-alcohol followed by a 42 L toluene vessel rinse. The vessel used for the DABCO solution make-up was again vacuum dried and to it charged 162.1 kg (850.2 mol) p-toluene sulfonyl chloride (tosyl chloride) followed by 542 L toluene. The mixture was stirred for 15 min at 10-16° C. to dissolve the solids (dissolution is endothermic) and then cooled to 2° C. The solution of tosyl chloride was then transferred to the solution of the ketal-alcohol and DABCO while keeping the reaction temperature <10° C. (addition performed over ˜3 h with a temperature range of −2 to +6° C.). To the vessel containing the tosyl chloride was added 43 L toluene as a vessel rinse. The reaction was stirred for 1 h at 3 to 4° C. and sampled for reaction completion (HPLC). The reaction completion showed 1 mg/mL ketal-alcohol remaining with excess tosyl chloride still present.

While the reaction completion sample was been analyzed, a 16,000 L glass-lined vessel was charged with 700 L potable water followed by 63.8 kg (759 mol) sodium bicarbonate. The mixture was stirred at ambient temperature to dissolve the solids. Once the tosylation reaction was deemed complete, the reaction mixture was added to the aqueous bicarbonate solution over 2 h at ambient temperature (jacket temperature setpoint of 20° C.) followed by 85 L toluene as a vessel rinse. The two-phase mixture was stirred for 2.3 h at 25-28° C. to hydrolyze the excess tosyl chloride and sampled for reaction completion (tosyl chloride not detected). The mixture was allowed to settle for 1 h at 29° C. and the lower aqueous layer separated. To the upper toluene layer was added 504 L potable water. The two-phase mixture was stirred for 30 min at 27° C. and allowed to settle for 1 h at 27° C. The lower aqueous layer was separated, combined with the sodium bicarbonate extract and discarded. The toluene solution of the Step 6 product was filtered through a 10-inch, 20-micron filter to a 12,000 L glass-lined reactor followed by 127 L toluene used as a vessel rinse. The filtered toluene solution was allowed to settle for at least 30 min followed by removal of any second phase water that was present before starting the subsequent feed distillation.

### Crystallization & Isolation

Approximately 1,018 L of the toluene solution of the (S)-chiral tosylate was transferred to a 2,000 L Hastelloy vessel. The solution was concentrated via a feed-distillation under reduced pressure (20 mm minimum pressure, vessel temperature ˜30-35° C. with a maximum bath temperature of 50° C.) until the volume of the (S)-chiral tosylate solution reached 353 L (no toluene rinse of the 12,000 L vessel was performed). The final strip volume prior to the heptane addition is important inasmuch as too much toluene will result in a lower product yield due to losses to the mother liquors.

Approximately half of the product solution was transferred to a 2,000 L glass-lined vessel (180 kg, density 1.07 kg/L) with the other half transferred back to the 12,000 L glass-lined feed vessel for storage. The 2,000 L glass-lined vessel (used as the Heinkel feed vessel) was too small to accommodate crystallizing the entire batch. It was therefore split and the crystallization performed in two parts. The batch was transferred through a mass flow meter to accurately determine how much of the batch was contained in each part. In this case, approximately 220 kg was contained in the second part necessitating that additional heptane above the standard charge be added for the second part crystallization.

To the Hastelloy distillation vessel was added two separate portions of 15 L toluene as a line rinse to each of the two glass-lined vessels. To the 2,000 L glass-lined vessel containing the first half of the batch was added 713.5 kg n-heptane at ambient temperature (20±5° C.) to crystallize the product. To each drum of n-heptane was added 8-10 drops of Octastat 5000 to increase the solvent conductivity. The charge rate of the n-heptane was limited to ≦8 kg/min. The product crystallization occurs during the heptane addition.

After the heptane addition and the product crystallization had occurred, the product slurry was cooled to −15° C. over 5.5 h and allowed to stir for 1 h at −15° C. The cool-down rate after the crystallization is not critical since the batch crystallizes during the heptane addition at ambient temperature. The product was isolated using a Heinkel and washed with pre-cooled n-heptane (<−10° C.) to give 108.1 kg product wet cake. As necessary, the mother liquors were used to rinse product from the crystallization vessel after the initial filtration sequence, especially for the second half isolation. After isolation was completed, the wet cake was loaded to a double-cone dryer and drying initiated (35° C. maximum bath temperature, full vacuum) while the second half of the batch was crystallized.

The second half of the batch was transferred from the 12,000 L glass-lined vessel to the 2,000 L glass-lined vessel used for the first half crystallization followed by 81.1 kg n-heptane as a vessel and line rinse. To the product solution was added an additional 790.6 kg n-heptane at 20±5° C. to crystallize the second half of the batch. After the heptane addition and the product crystallization had occurred, the product slurry was cooled to −15° C. over 10 h and allowed to stir for 1 h at −15° C. to −18° C. The product was isolated using a Heinkel and washed with pre-cooled n-heptane (<−10° C.) to give 140.2 kg product wet cake (248.3 kg total). After isolation was completed, the wet cake was loaded to the double-cone dryer containing the first half of the batch and drying restarted (35° C. maximum bath temperature, full vacuum). The product was dried until an LOD of ≦1.0% was achieved (0.00% LOD obtained on batch, LIMS 38-478512). The product was discharged from the dryer into double poly-lined fiber packs to give 224.1 kg (632.2 mol, 84.0% yield) of the (S)-chiral tosylate. A stirred sample of the mother liquors (˜3,100 L) was obtained which showed that ˜17.5 kg (49.5 mol, 6.6% yield based on 5.7 g/L assay of mother liquor sample) product was contained in the mother liquors.

### EXAMPLE 2

A mixture of 268 g THF and 177.7 g (1.00 equiv) of ethyl (3-chloro-4-(methylthio)phenylacetate were slowly added to a cold (<−15° C.) 20% solution of potassium tert-butoxide in THF (415.5 g, 1.02 equiv) and allowed to react over 2 hours at −15° C. to form a potassium enolate. A 1:1 solution mixture of 256.1 g (1.00 equiv) of (S)-(8,8-dimethyl-6,10-dioxaspiro[4.5]decan-2-yl)methyl 4methylbenzenesulfonate and 321 g THF was transferred slowly to the cold enolate reaction mixture solution. The alkylation reaction mixture was stirred at <0° C., warmed to 40° C. and then held at 40° C. until the reaction was complete. The THF was distilled off under vacuum and the resulting ester product extracted into 629 g of MTBE and 445 mL water. The bottom aqueous layer was extracted with another 100 g MTBE. The resulting two MTBE/product layers were combined.

The resulting intermediate alkylation product ester as a MTBE solution was directly utilized for a hydrolysis reaction by adding 69 g (1.2 equiv) of 50% aqueous sodium hydroxide. The mixture was heated to 50° C. and stirred until the reaction was complete. The MTBE was removed by distillation under vacuum. The product mixture was extracted into water by adding 613 g water and 612 g toluene. The bottom aqueous product layer was extracted again by adding 612 g toluene to remove any remaining by-products. The bottom aqueous product layer was pH adjusted using 35 g of a 50% citric acid solution to a pH of 8.5-9.0. The aqueous product layer was concentrated under vacuum to remove all residual toluene. This water mixture was carried into the subsequent oxidation reaction.

A solution of 4.8 g (0.02 equiv) sodium tungstate dihydrate catalyst in 12 g water was added to the reaction mixture. The reaction mixture was cooled to <15° C. and 209 g (1.2 equiv) of 30% aqueous hydrogen peroxide was added, maintaining the reaction mixture below 45° C. The mixture was pH adjusted to approximately 7.5 by adding small amounts (˜1-5 g) of a saturated aqueous sodium bicarbonate solution, if necessary. The reaction was stirred at 20° C. until the reaction was complete. The peroxide mixture was quenched using an aqueous solution of sodium sulfite. The quenched reaction mixture was then extracted by adding 407 g isopropyl acetate. The top organic layer was separated to remove by-products. The bottom aqueous product layer was pH adjusted to 4.7 using 140 g of a 50% solution citric acid and extracted by adding 610 g isopropyl acetate. The bottom layer was separated and extracted again with 558 g isopropyl acetate. The combined organic/product layers were distilled under vacuum to remove residual water. The resulting product slurry was heated to 65° C. with an additional 370 g of isopropyl acetate. The mixture was crystallized by slowly adding 498 g n-heptane. The slurry was concentrated under vacuum and an additional 632 g n-heptane added. The mixture was concentrated and slowly cooled to 10° C., aged, filtered, washed with n-heptane and dried to produce 229.7 g (73.8% yield) of (R)-2-(3-chloro-4-(methylsulfonyl)phenyl)-3-(8,8-dimethyl-6,10-dioxaspiro[4.5]-decan-2-yl)propanoic acid as a dry solid powder.

### EXAMPLE 3

### Epimerization Reaction

### Sodium Salt Formation

A 2000 L glass-lined reactor (vessel 1) was charged with 99.8 kg (232 mol, 1.00 equiv) of the reaction product 68 shown in FIG. 7 followed by 165.5 kg of denatured, 2B-3 ethanol. The mixture was stirred at 20° C. for 10 min. A solution of 112.5 kg 21% sodium ethoxide in ethanol was charged to vessel 1 followed by a line rinse of 5.1 kg denatured ethanol, 2B-3.

### Chiral Epimerization

The mixture was heated to 65° C. and stirred for ˜6 hours. The mixture was then cooled to 55° C. and sampled by chiral HPLC to determine the diastereomer ratio. After the age with sodium ethoxide in ethanol the percentage of the undesired isomer was expected to be <20% (2S, 3′R) relative to the (2R, 3′R) isomer by chiral HPLC analysis and, indeed, in the lab, 14.7% to 18.5% (2S, 3′R) was observed. This is as far as the epimerization can be taken in pure ethanol without significant production of aryl ethoxy and des-chloro impurities. While waiting for sample results, the mixture was heated back to 65° C.

While maintaining the vessel contents at 65° C., 573.1 kg of heptane was charged and the mixture stirred and aged for 2 h at 65° C. To add the heptane, ethanol is exchanged via a vacuum feed-strip with heptane and the reaction mixture aged for a minimum of 2 hours. The mixture was then cooled to 55° C. and sample. After the addition and age with n-heptane, the ratio of the (2R, 3′R) to (2S, 3′R) isomers was monitored along with the production of aryl ethoxy and des-chloro impurities using the chiral HPLC method. The bath temperature was set at 75° C. (the mixture refluxes at approximately 67° C.) and the reaction mixture was concentrated by atmospheric distillation to ˜450 L. The mixture was then cooled to 55° C. and sampled for final epimerization completion. After the distillation of n-heptane/ethanol, the percentage of the undesired isomer was expected to be <8% (2S, 3′R) relative to the (2R, 3′R) isomer by chiral HPLC analysis, and indeed 5.5 to 7.5% was observed. This is as far as the epimerization can be taken without significant production of aryl ethoxy and des-chloro impurities.

The mixture was cooled to 20° C. and 7.0 kg (117 mol, 0.5 equiv) of acetic acid was charged to another vessel, hereinafter vessel 2, followed by a line rinse of 2.0 L methanol. To vessel 2 was then charged 250.0 L methanol and the mixture stirred for 15 min. The mixture in vessel 2 was then transferred to vessel 1 while maintaining the vessel 1 contents at 20 (±5)° C. The reaction mixture was stirred for 15 min at 18° C. The mixture was then sampled to ensure the pH was between 6 and 8. The actual pH was 7.5. To vessel 2 was next charged 750 L methanol which was heated to 40° C. The reaction mixture in vessel 1 was atmospherically distilled while continuously charging methanol from vessel 2 through a ¼″ mass flow meter to maintain a constant volume in vessel 1. The mixture refluxes at about 57° C. About 880 L of distillate is collected. To vessel 2 was then charged 479.8 kg isopropyl alcohol which was heated to 50° C. The reaction mixture in vessel 1 was atmospherically distilled while continuously charging isopropyl alcohol from vessel 2 through a ¼″ mass flow meter to maintain a constant volume in vessel 1. The mixture will begin to reflux at ˜57° C. and this will increase to ˜71° C. The resulting slurry was slowly cooled over 2 h to 20° C. followed by aging at 20° C. for 1 h. The slurry was then sampled for final recrystallization completion. The slurry sample was filtered and the mother liquors analyzed for ratio of (2R, 3′R) to (2S, 3′R) isomers by chiral HPLC for comparison to reference batches (26.3 to 32.0% area norm (2R, 3′R) was observed) to ensure the correct volume had been reached to obtain a good yield of the product. The result was 32% area norm for the (2R, 3′R) isomer and 68% area norm for the (2S, 3′R) isomer by chiral HPLC analysis, which was consistent with reference batches.

### Isolation and Drying

To a Heinkel rinse vessel (vessel 3) was charged 272.9 kg isopropyl alcohol. The product slurry was filtered through the Heinkel centrifuge filter unit. Each spin was rinsed with a small quantity of the isopropyl alcohol from vessel 3. Each spin was initially filled with 3-5 kg of slurry and the product washed with ˜1 kg of isopropyl alcohol which resulted in about 1.5-2.0 kg of wet cake per spin. The combined isolated wet cakes were transferred to Krauss-Maffei conical dryer and dried under vacuum for ˜18 h at 40-45° C. to produce 94.7 kg (209 mol, 90.3% yield) of (2R, 3′R)-sulfone ketal acid, sodium salt [(2R, 3′R)-9)] isolated as a dry powder. The isolated material showed excellent purity by chiral HPLC and area normalized purity of 98.91%. However, the assay purity was only 93.6 wt % due to the presence of residual sodium acetate produced during neutralization with acetic acid. This salt was effectively removed in a subsequent hydrolysis step.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims.

What is claimed is:

1. A method of making a ketal alcohol, comprising the steps of:
providing a ketal acid;
contacting the ketal acid with a reducing agent in a hydrophobic solvent under conditions effective to convert the ketal acid to a ketal alcohol, said reducing agent comprising a hydride comprising one or more alkoxy moieties;
quenching the reduction reaction;
washing the hydrophobic solvent containing the ketal alcohol with one or more water washes, wherein at least a portion of the water washes include a portion of the ketal alcohol product; and
washing at least said portion of the one or more water washes with a back-extracting hydrophobic solvent to extract ketal alcohol from said portion into said back-extracting hydrophobic solvent.

2. The method of claim 1, wherein the ketal acid comprises a moiety of the formula —COOM, wherein M is selected from hydrogen, a monovalent organic substituent, or a cation.

3. The method of claim 2, wherein the cation is selected from sodium, potassium, lithium, ammonium, an aromatic cation, combinations of these, and the like.

4. The method of claim 3, wherein M is an aromatic cation of the formula
(Ar¹)ₚ—R²—N—(R^{o})_{q}⁺
wherein Ar¹ is a monovalent moiety comprising an aromatic ring; p is 1 to 4; each R^{o} is independently hydrogen and/or an achiral or chiral monovalent moiety that may be substituted or unsubstituted, or linear, branched, or cyclic; q is 0 to 3; and p+q is 4.

5. The method of claim 1, wherein the ketal acid is converted to a ketal alcohol according to a reaction scheme in accordance with FIG. 3, FIG. 4, or FIG. 5.

6. The method of claim 1, further comprising the step of extracting the ketal alcohol into one or more organic phases.

7. The method of claim 1, wherein the contacting occurs in a solvent comprising toluene.

8. The method of claim 1, wherein the providing step comprises the steps of
providing an admixture comprising a ketal salt comprising a chiral, aromatic cation and a solvent comprising toluene;
contacting the admixture with an aqueous acid under conditions to form a ketal acid; and
extracting the ketal acid into an organic phase comprising the acid and toluene.

9. A method of making a pharmacologically active compound, comprising the steps of:
providing a ketal acid;
contacting the ketal acid with a reducing agent in a hydrophobic solvent under conditions effective to convert the ketal acid to a ketal alcohol, said reducing agent comprising a hydride comprising one or more alkoxy moieties;
quenching the reduction reaction;
washing the hydrophobic solvent containing the ketal alcohol with one or more water washes, wherein at least a portion of the water washes include a portion of the ketal alcohol product;
washing at least said portion of the one or more water washes with a back-extracting hydrophobic solvent to extract ketal alcohol from said portion into said back-extracting hydrophobic solvent; and
using the ketal alcohol to make the pharmacologically active compound.

10. The method of claim 9, wherein the pharmacologically active compound comprises the compound of the Formula 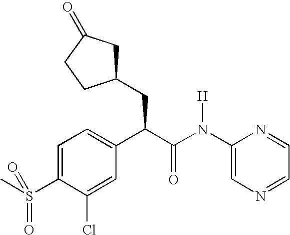
or a solvate thereof.
